(19) United States
(12) Patent Application Publication
(10) Pub. No.: US 2008/0114402 A1
(43) Pub. Date: May 15, 2008

(54) Devices and Methods for Correcting a Spinal Deformity

(75) Inventors: Randal Betz, Ocean City, NJ (US); Fred J. Molz, Birmingham, AL (US)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/558,559

(22) Filed: Nov. 10, 2006

(51) Int. Cl. A61B 17/88

(52) U.S. Cl. 606/279

(57) ABSTRACT

The present application is directed to devices and methods for correcting a spinal deformity. One exemplary method comprises initially inserting a device into an interior of a vertebral member. The device may be a deformable material, or may be a mechanical apparatus. The device is then expanded in size such that it contacts against the interior of the vertebral member. The expanded device applies a force on the interior of the vertebral member that causes the member to expand from a first size to a second size. The process may include insertion into a single vertebral member, or multiple vertebral members along the spine.

### BACKGROUND

The present application is directed to devices and methods for correcting spinal deformities and, more particularly, to methods and devices that apply a force or pressure to the interior of the vertebral member.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various deformities may affect the normal alignment and curvature of the vertebral members. Scoliosis is one example of a deformity of the spine in the coronal plane, in the form of an abnormal curvature. While a normal spine presents essentially a straight line in the coronal plane, a scoliotic spine can present various lateral curvatures in the coronal plane. The types of scoliotic deformities include thoracic, thoracolumbar, lumbar or can constitute a double curve in both the thoracic and lumbar regions. Scheurmann's kyphosis is another deformity that affects the normal alignment and curvature.

Many prior methods and devices have disclosed measures for correcting the deformities. These measures include applying a force to the exterior of the vertebral members to correct the curvature. One example is a vertebral rod that is attached along the exterior of the vertebral members to apply a force to move the vertebral members back to the normal alignment.

### SUMMARY

The present application is directed to devices and methods of correcting a spinal deformity. One exemplary method comprises initially inserting a device into an interior of a vertebral member. The device may be a deformable material, or may be a mechanical apparatus. The device is then expanded in size such that it contacts against the interior of the vertebral member. The expanded device creates an intraosseous expansile pressure or force, referred to as intraosseous expansile force, within the interior of the vertebral member that causes the member to expand from a first size to a second size. The process may include insertion into a single vertebral member, or multiple vertebral members along the spine.

### BRIEF DESCRIPTION OF THE DRAWINGS

FIG. 1 is a schematic view of a deformed spine according to one embodiment.

FIG. 2 is a schematic coronal view of material within a vertebral member according to one embodiment.

FIG. 3 is a schematic coronal view of expanded material within a vertebral member according to one embodiment.

FIG. 4 is a schematic axial view of a cannula extending into a void in a vertebral member according to one embodiment.

FIG. 5 is a schematic view of device that includes a shell and material according to one embodiment.

FIG. 6 is a schematic coronal view of materials within voids in a vertebral member according to one embodiment.

FIG. 7 is a schematic coronal view of materials within a void in a vertebral member according to one embodiment.

FIGS. 8A and 8B are schematic coronal views of material in a vertebral member with corrective osteotomies according to one embodiment.

FIG. 9 is a schematic coronal view of a syringe inserting material into a vertebral member according to one embodiment.

FIGS. 10A and 10B are schematic coronal views of a device in a vertebral member with a corrective osteotomy according to one embodiment.

### DETAILED DESCRIPTION

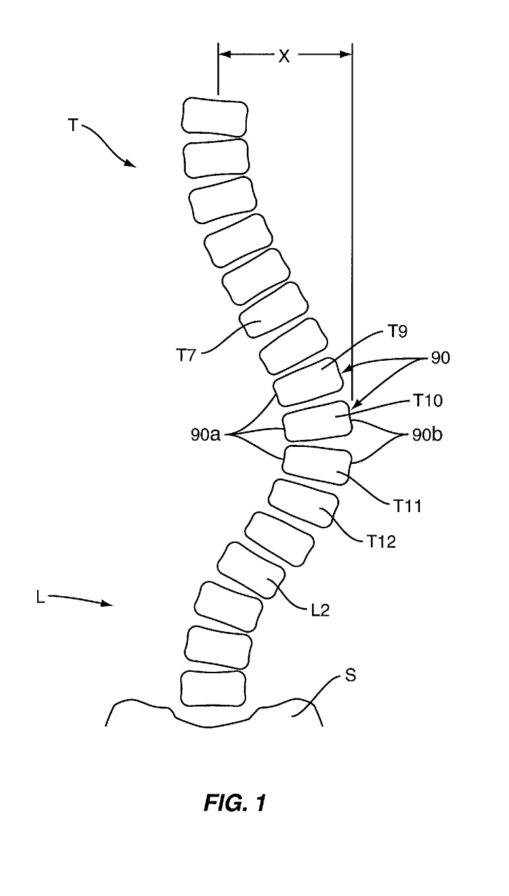

FIG. 1 illustrates a patient's spine that includes a portion of the thoracic region T, the lumbar region L, and the sacrum S. This spine has a scoliotic curve with an apex of the curve being offset a distance X from its correct alignment in the coronal plane. The spine is deformed laterally so that the axes of the vertebral members 90 are displaced from the sagittal plane passing through the midline of the patient. In the area of the lateral deformity, each of the vertebral members 90 includes a concave side 90a and a convex side 90b. The present application is directed to devices and methods for applying an intraosseous expansile force to one or more of the vertebral members 90 to correct the spinal deformity. Devices and methods apply an intraosseous expansile force to the concave side 90a of the vertebral members 90. The force causes the vertebral members 90 to elongate and move the overall shape of the spine towards a normal configuration.

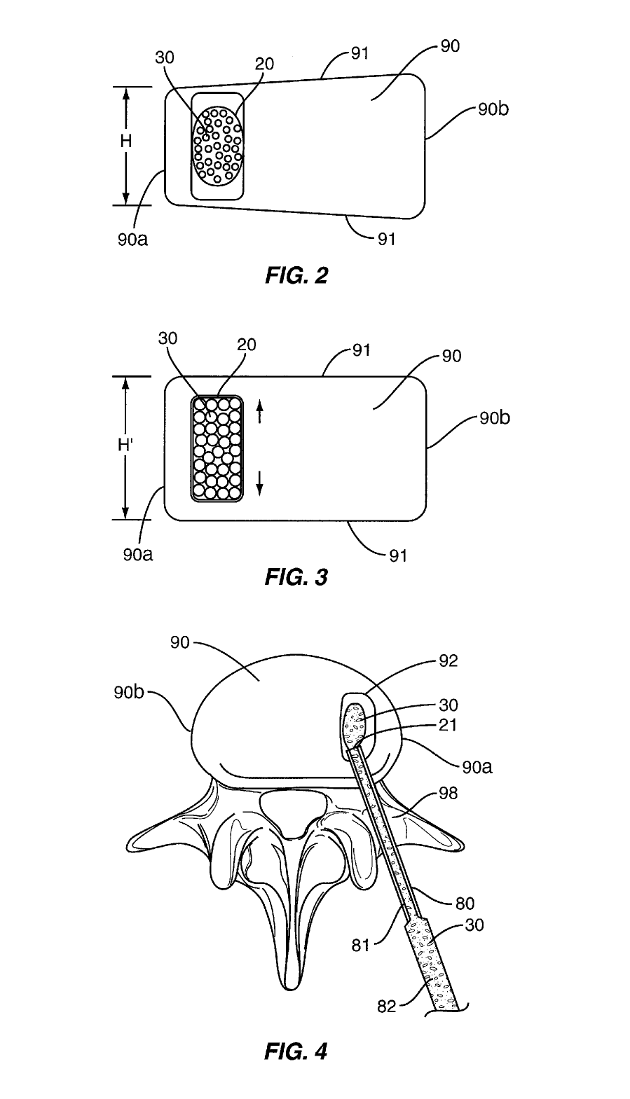

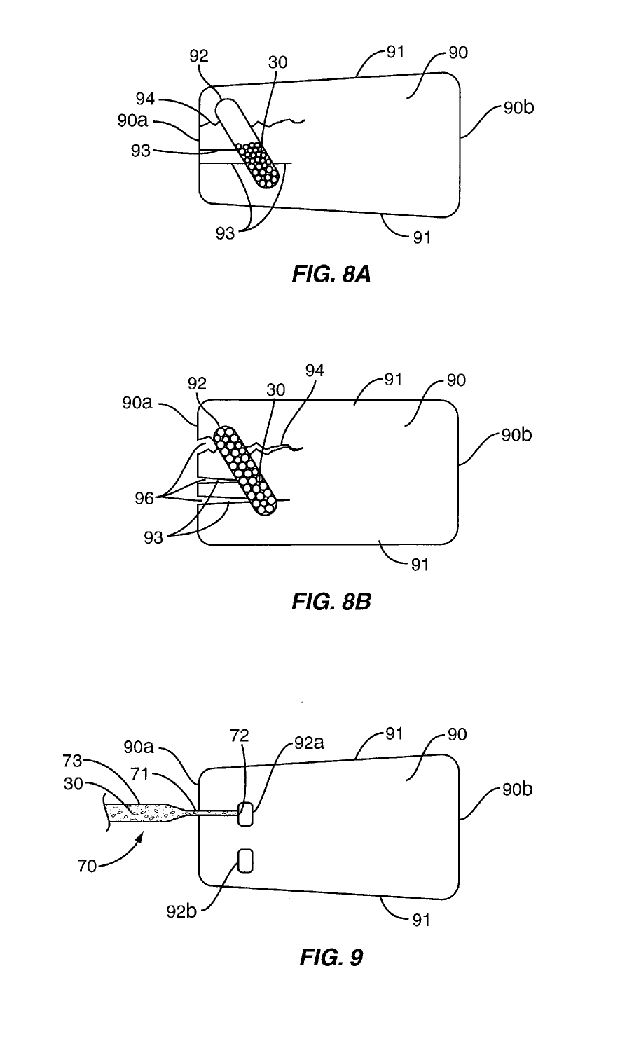

FIG. 2 illustrates one embodiment with a void 92 formed within the interior of the vertebral member 90. Void 92 is formed at the concave side 90a away from the convex side 90b and between the endplates 91. Void 92 is mainly formed within the cancellous bone, although it may also extend into the cortical rim along the lateral edges and endplates 91. Void 92 may include a height to contact the interior of the endplates 91 as illustrated in FIG. 2, or may be spaced inward from one or both of the endplates 91 as illustrated in void 92b of FIG. 6. The void 92 may include a variety of heights, widths, and sizes depending upon the context of use. Further, multiple voids may be formed within the vertebral member 90 as illustrated in FIG. 6. In some embodiments, voids 92 are aligned in a vertical manner within the vertebral member 90 as illustrated in FIG. 6. Voids may also be positioned at an angled orientation within the vertebral member 90 as illustrated in FIGS. 8A and 8B.

A shell 20 that holds an expandable material 30 is positioned within the void 92. The shell 20 may be constructed of a flexible material and may have a predefined shape, or may be amorphous. Shell 20 may also be expandable to accommodate the material 30 as it increases in size. The shell 20 may further be hydrophilic and/or permeable to permit liquid to pass through to the interior and contact the material 30. Shell 20 may be constructed from a variety of materials. Examples include but are not limited to various polymeric materials, such as aliphatic or aromatic polycarbonate-based and non-polycarbonate-based polyurethanes, polyethylene terephthalates, polyolefins, polyethylene, polycarbonate, ether-ketone polymers, polyurethanes, nylon, polyvinyl chloride, acrylic, silicone, and combinations thereof. The shell 20 may further be reinforced with woven or non-woven textile materials. Examples of suitable reinforcement materials include those that are polymeric and metallic in nature. The shell 20 may also be elastic, but impermeable to the activation fluid. Since the activation fluid cannot leak out of the shell 20, a prescribed amount of fluid can be introduced into the shell 20 at the time of placement to control the magnitude of the intraosseous expansile force.

Material 30 is positioned within the shell 20 and is activated to expand from a first state with a first size to a second state with an enlarged size. The material 30 in the first state may be malleable to facilitate insertion and positioning within the void 92. The material 30 in the first state may range from an injectable liquid, to a foam, to a visco-elastic solid. Upon activation, the material 30 begins to transform and expand to the second state. Further, the material 30 may transform to a hardened state. The term “hardened” and the like refers to materials and combination of materials that can solidify, in situ, at the tissue site, to assume a load bearing capacity.

Material 30 may be homogeneous with the same chemical and physical properties throughout, or heterogeneous. A variety of materials may be used and may include silicones, polyurethanes, silicone-polyurethanes, polyvinyl chlorides, polyethylenes, styrenic resins, polypropylene, polyolefin rubber, PVA, protein polymers, thermoplastic polyesters, thermoplastic elastomers, polycarbonates, acrylonitrile-butadiene-styrene resins, acrylics, nylons, styrene acrylonitriles, cellulosics, DBM, PMMA bone cement, tissue growth factor, epoxy, calcium phosphate, calcium sulfate, and resorbable polymers such as PLA, PLDLA, and POLYNOVO materials. The material may also include a pharmaceutical composition comprising one or more biological response modifiers. Material 30 may further include an opaque additive, such as barium sulfate, that will be visible on an X-ray. Examples of various materials are disclosed in U.S. patent application Ser. No. 11/392,030 filed on Mar. 29, 2006 and entitled Transformable Spinal Implants and Methods of Use, herein incorporated by reference in its entirety.

FIG. 2 illustrates an embodiment with the material 30 in the first state, such as upon initial placement into the void 92. The shell 20 with the material 30 may completely fill the void 92, or only a portion of the void 92. At some point, the material 30 is exposed to an activation event and begins to change from the first state to the expanded second state. FIG. 3 illustrates an embodiment with the material 30 in the second state. The material 30 has expanded to substantially fill the void 92 and apply an intraosseous expansile force to the vertebral member 90. The force results in the overall height of the concave side 90a increasing to height H′ from height H as illustrated in FIG. 2. In some embodiments, the height of the concave side 90a is increased to be about equal to the height of the convex side 90b. In other embodiments, the height of concave side 90a is increased to be greater than the convex side 90b. In still other embodiments, the height of the concave side 90a is increased, but is still less than the convex side 90b.

The shell 20 and material 30 may be inserted into the interior of the vertebral member 90 in a variety of manners. FIG. 4 illustrates one embodiment with percutaneous access with a posterior approach through the pedicle 98 and into the concave side 90a of the vertebral member 90. Initially, a cutting tool (not illustrated) is used for forming an opening through the vertebral member 90 and into the concave side 90a. A cannula 80 may then be inserted into the opening to provide access to the concave side 90a. The same cutting tool that formed the opening, or a second tool, may then be inserted through the cannula 80 to create the void 92. One embodiment of a cutting tool is disclosed in U.S. Pat. No. 6,923,813 herein incorporated by reference. In another embodiment, the void 92 is formed with an inflatable balloon. The balloon is inserted through the cannula 80 and into the concave side 90a. The balloon is then inflated to compress at least a portion of the cancellous bone to form the void 92. Embodiments of balloons are disclosed in U.S. Pat. No. 6,923,813 and U.S. Patent Application No. 2004/0092948, each herein incorporated by reference.

The shell 20 is then inserted through the cannula 80 and into the void 92. In one embodiment, the shell 20 is pre-filled with the material 30 prior to insertion into the void 92. The shell 20 and material 30 are deformable to fit within the cannula 80 and be inserted into the void 92. In another embodiment, the shell 20 is initially inserted into the void 92. After the shell 20 is inserted, the material 30 is moved through the cannula 80 and inserted into the shell 20. As illustrated in FIG. 4, the material 30 may be stored in a reservoir 82 prior to being moved through a second cannula 81 and into an opening 21 in the shell 20. After the shell 20 is filled, the opening 21 is sealed and the cannula 80 and second cannula 81 are removed from the patient. Bone fusion material or bone cement may be introduced into the opening in the vertebral member 90 to facilitate complete closure and ultimate bone union.

The shell 20 with the material 30 in the first state has a first height H as illustrated in FIG. 2. An activation event occurs that begins the transformation of the material 30 towards the expanded, second state. In one embodiment, shell 20 is constructed of a permeable material. The activation event comprises fluid from the patient that enters through the shell 20 and contacts the material 30. This contact causes the material to expand in size to the second state as illustrate in FIG. 3. The growth causes an outward intraosseous expansile force on the vertebral member 90 as illustrated by the arrows in FIG. 3. The internal force induces bony growth on the vertebral member 90 that results in an increase in the size of the vertebral member 90. In this embodiment, the height of the vertebral member 90 increases from height H when the material 30 is in the first state, to height H′ with the material 30 in the second state.

In one embodiment, contact with fluid begins the expansion. The contact may occur because the shell 20 is constructed of a hydrophilic material. In another embodiment, fluid is introduced into the shell 20 during the insertion process. Fluid may be injected into the shell prior to insertion into the void 92, or may be inserted after insertion. In one specific embodiment, fluid is injected into the opening 21 after the material 30 has been inserted into the shell 20.

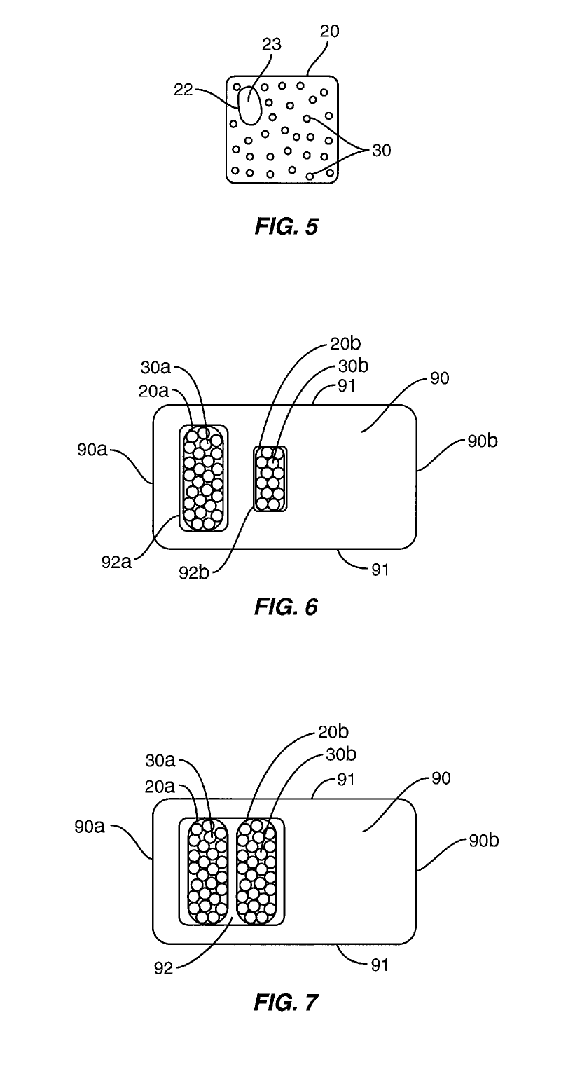

FIG. 5 illustrates another embodiment of a device with material 30 positioned within the shell 20. A second shell 22 that contains fluid 23 is also positioned within the shell 20. Second shell 22 is ruptured causing the fluid 23 to mix with the material 30 and begin the activation. The second shell 22 may be ruptured during insertion into the void 92, or may be ruptured after insertion.

The amount of time for the material 30 to expand to the second state may depend upon the material itself and the extent of the activation event. Various materials will increase in size at a more rapid pace than others. Also, a stronger activation event may cause the growth to occur at a more rapid pace. By way of example, the growth may be faster when a greater amount of fluid is applied to the material 30. Smaller amounts of fluid may result in slower expansion.

The amount of time for the material 30 to fully change into a hardened second state may vary. In some embodiments, the material 30 takes days and months to expand fully to the second state. In other embodiments, less time is necessary for full expansion and hardening. In one embodiment, the material 30 cures to a hardened second state within about two minutes to about six hours after activation. The longer expansion times are aimed at altering bony growth. In some embodiments, shorter expansion times would be required when a fracture or localized weakening is used to expand the height of the vertebral member 90 and, after expansion, some stability may be required for healing.

In one embodiment, two different types of material are positioned within the shell 20. The materials may be activated through different events, and may have different expansion rates. A first activation event may occur that causes the first material to begin expansion. At some time thereafter, a second activation event occurs that causes the second material to expand. The amount of time between the two activation events may vary depending upon the context of use.

Various types of activation events may cause the material 30 to change to the second state. These events may include a chemical reaction, thermal reaction such as with a heat gun or autoclave chamber, photo reaction such as visible, ultra-violet, or infrared light, radiation source such as an X-ray device or fluoroscopy arm, electrical source such as a battery or source that emits AC or DC electrical current, light energy including ultraviolet or infrared light sources, and physical energy such as pressure or impact force.

The vertebral member 90 may be treated in a variety of different methods to grow the concave side 90a. In one embodiment, two or more voids 92 may be formed within the vertebral member 90. FIG. 6 illustrates a single vertebral member 90 with a first void 92a with a first shell 20a and material 30a, and a second void 92b with a second shell 20b and material 30b. FIG. 7 illustrates another embodiment with a single void 92 sized to contain multiple shells 20a, 20b and materials 30a, 30b.

In embodiments with multiple shells 20, each shell 20 may include a different type and/or amount of material 30 to apply a different internal pressure to the vertebral member 90. Using FIG. 6 as an example, shell 20a may include a first material 30a with a first expansion factor. Second shell 20b may include material 30b with a different second expansion factor. This causes the first shell 20a to apply a first internal force to the vertebral member 90 that is different than the second shell 20b. In one embodiment, the first shell 20a that is positioned in closer proximity to the lateral edge of the concave side 90a may apply a greater force than shell 20b which is more centrally located.

One or both of the shell 20 and material 30 may be bioresorbable. In one embodiment, the shell 20 is a bioresorbable non-porous (sheet or film) or a bioresorbable porous (braided fibers) shell. The material 30 is a precursor of resorbable polymer that polymerizes, cures or crosslinks in situ.

In some embodiments, the material 30 is placed within a shell 20. In other embodiments, the material 30 is placed directly into the void 92 without a shell 20. FIG. 8A illustrates an embodiment with the material 30 positioned within the void 92 and directly in contact with the vertebral member 90. The material 30 is activated and grows into the expanded, second state as illustrated in FIG. 8B. The activation events may be the same for the material 30 whether it is contained within a shell 20 or placed directly within the void 92.

In some embodiments, the vertebral member 90 is weakened to facilitate growth. The weakening may occur through cuts or fractures. FIGS. 8A and 8B illustrate an embodiment with one or more cuts 93 made into the concave side 90a. The cuts 93 may include a variety of lengths, and may extend through just the cortical rim, or may also expand into the cancellous bone. The cuts 93 may further extend inward from the lateral edge to the opposite side of the void 92. The number and size of cuts may vary depending upon the growth requirements. Likewise, one or more fractures 94 may likewise be made in the vertebral member to again facilitate the growth. The number, size, and spacing of the fractures 94 may vary depending upon the desired growth.

After the material 30 has expanded and the vertebral member 90 has grown, the cuts 93 and/or fractures 94 may expand as illustrated in FIG. 8B. This expansion may cause gaps 96 to form. These gaps 96 may naturally heal and close. Alternatively, or in addition to the natural healing, bone fusion material or bone cement (not illustrated) and the like may be inserted into the gaps 96 to strengthen the vertebral member 90.

FIG. 9 illustrates another embodiment for inserting the material 30 into the vertebral member 90. A syringe 70 is used for inserting the material 30 into the vertebral member 90. The syringe 70 includes a reservoir 73 for holding the material 30 and a cannula 71 sized to be inserted into the vertebral member 90. The cannula 71 includes an opening 72 that is positioned within the void 92a formed within the vertebral member 90. The syringe 70 may insert material 30 into one or more separate locations within the vertebral member 90. In this embodiment, material 30 is inserted into first and second voids 92a, 92b.

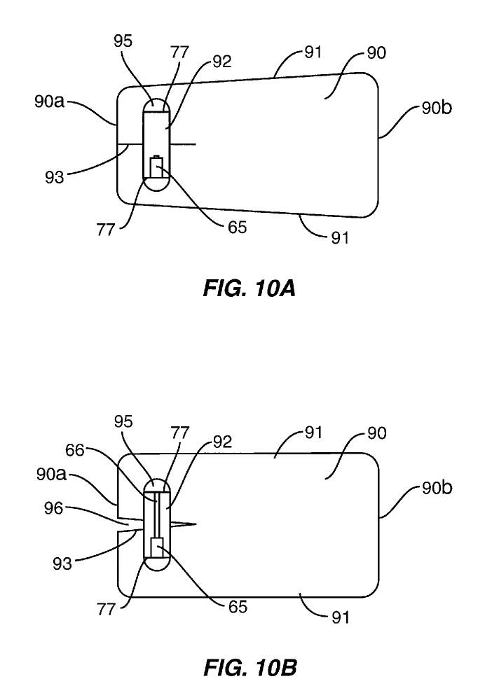

A mechanical device may further be inserted within the vertebral member 90 to apply the intraosseous expansile force. An example of an expandable device includes two threaded plates separated by a turnbuckle. The plates include left-handed and right-handed posts that connect with the turnbuckle with rotation of the turnbuckle spacing the plates apart. FIG. 10A illustrates an embodiment with a mechanical device 65 positioned within the void 92. The device 65 may be positioned in a first orientation that is sized to be minimally invasive to facilitate insertion into the vertebral member 90. In one embodiment, the device 65 is sized to fit within the opening forming by the cutting tool as illustrated in FIG. 4. The device 65 may then be expanded to a second orientation as illustrated in FIG. 10B. An arm 66 extends outward from the device 65 such that the device spans the height of the void 92. The device 65 applies an intraosseous expansile force to the vertebral member to cause the height of the concave side 90a to increase. The device 65 may expand to the second orientation through a variety of means, such as a rotational force that rotates a gear causing the arm 66 to move. In another embodiment, fluid is pumped into the device 65 causing the arm 66 to move in and out accordingly.

In some embodiments, the device 20, 65 directly contacts the vertebral member 90. In some embodiments, the device 20, 65 contacts the inner edge of the cortical rim. In other embodiments as illustrated in FIGS. 10A and 10B, one or more supports 77 are positioned within the vertebral member 90 to provide a contact surface. As illustrated in the embodiment of FIGS. 10A and 10B, supports 77 are mounted within the upper and lower reaches of the void 92. The supports 77 are fixedly positioned within the vertebral member 90 and include a contact surface that faces towards the device 65. During expansion, the device 65 contacts the supports 77 to apply the force to the vertebral member 90.

In some embodiments, after a predetermined time period of being activated and changing into the second state, the material 30 may become solid and support the vertebral member 90. In other embodiments, the material 30 remains in the same form as in the first state.

Devices 20, 65 may be inserted into one or more of the vertebral members 90 located along the spine to correct the spinal deformity. Using FIG. 1 as an example, one or more devices 20, 65 may be inserted within vertebral member T10. Treatment of the vertebral member T10 may be sufficient to correct the spinal deformity. However, additional devices 20, 65 may be inserted in other vertebral members such as T9 and T7 which are on the superior section of the deformity, and T11, T12, and L2 which are on the inferior section. The number of vertebral members that are treated and the extent of growth of the vertebral members may vary depending upon the individual patient.

It should be understood that the spinal deformity depicted in FIG. 1 is but one of many types of spinal deformities that can be addressed by the devices and techniques of the present application. Most commonly the devices and methods are expected to be used for either primary thoracic or thoracolumbar curves. They can be used for correction of the thoracic curve as an isolated curve, or the lumbar curve as an isolated curve. The devices may further be used in combination with the shortening of the opposite side of the vertebral member 90. Furthermore, anterior treatment of Scheurmann's kyphosis is also contemplated, as is correction of compression deformities in the sagittal plane.

In one embodiment, shell 20 is filled with a gas or fluid. The gas and fluid are pressurized to apply an intraosseous expansile force. The shell 20 is pressurized at the time of insertion and there is no active expansion period. The pressure within the shell 20 may be periodically increased or decreased through percutaneous injections that remove or add gas or fluid as necessary.

One embodiment includes accessing the spine from a posterior approach to the spine. Other applications contemplate other approaches, including posterior, postero-lateral, antero-lateral and lateral approaches to the spine, and accessing the various regions of the spine, including the cervical, thoracic, lumbar and/or sacral portions of the spine.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The methods and devices disclosed herein may be carried out in other specific ways than those herein set forth without departing from the scope and essential characteristics of the application. In one embodiment, the material is forced into the vertebral member 90 without forming a void 92 prior to insertion. The present embodiments are, therefore, to be considered in all respects as illustrative and not restrictive, and all changes coming within the meaning and equivalency range of the appended claims are intended to be embraced therein.

What is claimed is:

1. A method of correcting a spinal deformity comprising the steps of:
inserting a device into an interior of a vertebral member;
expanding a size of the device and contacting the device against the interior of the vertebral member; and
applying an intraosseous expansile force on the vertebral member and causing the vertebral member to expand from a first size to a second size.

2. The method of claim 1, wherein the step of inserting the device into the interior of the vertebral member comprises positioning the device at a concave side of the vertebral member.

3. The method of claim 1, further comprising forming a void within the interior of the vertebral member and inserting the device into the void.

4. The method of claim 1, wherein the step of inserting the device into the interior of the vertebral member comprises positioning the device within a cortical rim of the vertebral member.

5. The method of claim 1, wherein the step of causing the vertebral member to expand from the first size to the second size comprises increasing a height of a concave side of the vertebral member.

6. The method of claim 1, further comprising applying an activation event to the device and expanding the size of the device.

7. The method of claim 1, further comprising positioning a second device within the interior of the vertebral member and expanding the second device and applying a second intraosseous expansile force.

8. The method of claim 1, wherein the step of inserting the device into the interior of the vertebral member comprises inserting an expandable material into the interior while the material is in a first reduced state.

9. The method of claim 1, further comprising weakening a portion of the vertebral member and causing the weakened portion to increase from a first height to a second height.

10. The method of claim 1, further comprising weakening a concave side of the vertebral member and causing the concave side to increase from a first height to a second height.

11. The method of claim 1, wherein after the step of inserting the device into the interior of the vertebral member, percutaneously accessing the device and inserting additional material into the device.

12. A method of correcting a spinal deformity comprising the steps of:
forming a void within a concave side of an interior of a vertebral member;
inserting a device into the void;
expanding the device and contacting the device against superior and inferior sections of the void;
applying an intraosseous expansile force against the superior and inferior sections of the void; and
causing the concave side of the vertebral member to expand from a first height measured between inferior and superior endplates to a second greater height.

13. The method of claim 12, wherein the step of forming the void within the concave side of the vertebral member comprises accessing the vertebral member from a posterior approach and forming an opening through a pedicle and into the interior of the vertebral member.

14. The method of claim 12, wherein the step of inserting the device into the void comprises inserting an expandable material into the void while the material is in a first reduced state.

15. The method of claim 12, wherein the step of inserting the device into the void comprises inserting an expandable mechanical device into the void while the mechanical device is in a first reduced state.

16. The method of claim 12, further comprising weakening the concave side of the vertebral member to allow expansion from the first height to the second height.

17. The method of claim 12, further comprising inserting a second device into the void and expanding the second device and applying a second intraosseous expansile force against the superior and inferior sections of the void.

18. The method of claim 17, further comprising expanding the device and applying the intraosseous expansile force against the superior and inferior sections of the void prior to expanding the second device.

19. The method of claim 12, further comprising forming a second void within the interior of the vertebral member and inserting a second device into the second void and expanding the second device to apply a second intraosseous expansile force within the interior of the vertebral member.

20. A method of correcting a spinal deformity comprising the steps of:
forming a void within a concave side of an interior of the vertebral member;
inserting a device into the void while the device is in a first state;
activating the device;
expanding the device within the void from the first state to a second state and applying an intraosseous expansile force to the vertebral member; and
causing the concave side of the vertebral member to expand from a first height measured between inferior and superior endplates to a second greater height.

21. The method of claim 20, wherein the step of inserting the device into the void comprises inserting an expandable material into the void while the material is in the first state.

22. The method of claim 20, further comprising inserting a support into the void and expanding the device within the void to contact the support and apply the intraosseous expansile force against the support.

23. The method of claim 20, wherein the step of inserting the device into the void comprises inserting a mechanical device into the void while the device is in a first reduced size.

24. The method of claim 20, further comprising fracturing the vertebral member and allowing the concave side of the vertebral member to grow to the second height.

25. The method of claim 20, further comprising cutting the concave side of the vertebral member prior to causing the concave side of the vertebral member to expand to the second height.

26. The method of claim 20, further comprising maintaining the device in the second state after the concave side expands to the second height.

27. The method of claim 20, further comprising inserting a second device into a second vertebral member and causing the second vertebral member to expand to an enlarged size.

28. The method of claim 20, wherein after the step of expanding the device within the void from the first state to a second state and applying an intraosseous expansile force to the vertebral member, adding material into the device an increasing the intraosseous expansile force.

29. A method of correcting a spinal deformity comprising the steps of:
forming a void within a concave side of an interior of the vertebral member;
inserting a material into the void while in a first state;
exposing the material to an activation event;
expanding the material within the void from the first state to a second state and applying an intraosseous expansile force to the vertebral member; and
causing the concave side of the vertebral member to expand from a first height measured between inferior and superior endplates to a second height.
